(19) United States
(12) Patent Application Publication
(10) Pub. No.: US 2024/0189498 A1
(43) Pub. Date: Jun. 13, 2024

(54) SURGICAL SUCTION VENTING SYSTEM

(71) Applicant: W. L. Gore & Associates GmbH, Putzbrunn (DE)

(72) Inventors: Nicole Gruen, Putzbrunn (DE); Wolfgang Hohma, Putzbrunn (DE); Peter Lang, Putzbrunn (DE); Isabel A. Willim, Putzbrunn (DE)

(21) Appl. No.: 18/286,480

(22) PCT Filed: Apr. 8, 2022
(86) PCT No.: PCT/EP2022/059469

§ 371 Date: Oct. 11, 2023

Related U.S. Application Data
(60) Provisional application No. 63/173,569, filed on Apr. 12, 2021.

(51) Int. Cl. A61M 1/00; B01D 46/00; B01D 46/12; B01D 46/62

(52) U.S. Cl. CPC A61M 1/784; B01D 46/0012; B01D 46/12; B01D 46/62; A61M 2205/7509; A61M 2205/7518; A61M 2205/7536; A61M 2205/7545

(57) ABSTRACT

A filter system for use with a suction canister or suction bag is disclosed, where the filter system includes a first filter and a second filter for retention of particles, smoke aerosols, liquids, aerosolized viruses, and aerosolized bacteria, while maintaining high airflow and liquid retention. The first filter and the second filter are each multi-layered for performance. In various examples, the first and second filters are configured to integrate directly with the vacuum port of a suction canister lid.

### CROSS-REFERENCE TO RELATED APPLICATION

This application is a national phase application of PCT Application No. PCT/EP2022/059469, internationally filed on Apr. 8, 2022, which claims the benefit of provisional Application No. 63/173,569, filed Apr. 12, 2021, which are herein incorporated by reference in their entireties for all purposes.

### FIELD

The present disclosure relates to filter systems, such as those used for medical equipment. Specifically, various examples relate to a filter system for a medical suction canister.

### BACKGROUND

During medical procedures, including surgery, suction canister systems are used to collect fluids, including blood and other bodily fluids, and rinsing solutions, such as saline or Ringer's solution. Such fluids may accumulate within the body during a medical procedure, and must be removed, contained, and disposed of following the procedure. A conventional canister system may include a cylindrical canister closed by a cover or lid. The cylindrical canister may include a liner, or the canister may be a soft-sided container, or bag, having a cover or lid. The lid includes a vacuum port to operably couple the canister to a vacuum to create a sub-atmospheric pressure within the canister. A collection tube may also be coupled to a patient port of the lid, where a vacuum is formed at the lid-end of the collection tube to create suction. Various attachment or connection elements may also be included with the canister system, including an outlet, or “ortho” port, having a wide riser portion, a capped large access port, or a float valve configured to prevent suction of fluid into the vacuum.

Often, the vacuum operably coupled to the canister is part of a vacuum system that is common to several areas of a corresponding facility. As such, it is important that entry of foreign objects, particles, aerosols, surgical smoke, bacteria, viruses, and fluids into the vacuum system is minimized to avoid contamination of the remainder of the system. In fluid collection systems, the fluids and/or materials in said fluids may vaporize and become airborne under the influence of the vacuum system. To mitigate contamination, filter elements may be used in the vacuum ports of canister systems, collectively known as an aerosol trap. Aerosol traps are conventionally assembled on the canister side of the vacuum port, all of which require individual handling of the filter assembly components. Conventional canisters are not configured to effectively filter the aforementioned contaminants. Instead, such filtration is attempted using additional accessories that are connected between the canister and the vacuum pump. These additional accessories must be acquired, stored, selected, assembled, used, and disposed. The assembly process and the disassembly process are time-consuming and labor-intensive due to the level of detail required to properly assemble the filter assembly within the canister to reach the appropriate level of filtration to prevent contamination of the vacuum system.

### SUMMARY

In a first Aspect of the disclosure, a filter system is disclosed. The filter system includes a housing defining an interior chamber with a chamber opening and an outlet port disposed on a base opposite of the chamber opening; a first support member coupled to the housing; a first filter positioned over the chamber opening of the housing; and a second filter positioned between the first support grid and the first filter, wherein a liquid reservoir is defined by the first filter and the second filter.

In a second Aspect of the disclosure, a filtering assembly is disclosed. The filtering assembly includes a lid defining a port and a filter system integrated into the port. The filter system includes a first filter defining a thickness of about 120 microns to about 2000 microns and a second filter having a liquid entry pressure greater than or equal to 75 kPa for 10 minutes at a surface tension of 55.5 mN/m and maintain an airflow of greater than 20 litres/min at a pressure drop of 11.5 kPa at an active area of 9.3 cm². The first filter includes a first layer and a second layer, wherein the first layer is comprised of a different material than the second layer.

In a third Aspect of the disclosure, a filter system is disclosed. The filter system includes a housing. The housing includes a base, a port extending from a first side of the base and defining an outlet, a first wall extending form a second side of the base, and a second wall extending from the second side of the base, wherein the first wall and the second wall define a groove. The filter system also includes a first support member including an upper sidewall configured to be received within the groove of the housing to couple the support member to the housing; a splash guard, defining an inlet and an outlet, coupled to the first support member; a first filter positioned at the outlet of the splash guard; a second support member positioned between the first filter and the first support member; and a second filter positioned between the first support member and the second support member.

In various Aspects of the disclosure, the second filter includes a liquid entry pressure greater than or equal to 75 kPa for 10 minutes at surface tension of 55.5 mN/m and maintain an airflow of greater than 20 litres/min at a pressure drop of 11.5 kPa at an active area of 9.3 cm². In various Aspects of the disclosure, the liquid reservoir has a thickness from 0.04 millimeters to 2 millimeters. In various Aspects of the disclosure, the liquid reservoir has a thickness from 2 millimeters to 15 millimeters. In various Aspects of the disclosure, the liquid reservoir has a thickness from 15 millimeters to 80 millimeters. In various Aspects of the disclosure, the first filter defines a thickness of about 120 microns to about 2000 microns. In various Aspects of the disclosure, the liquid reservoir comprises a third layer of the first filter. In various Aspects of the disclosure, wherein the liquid reservoir comprises a second support member positioned between the first filter and the second filter so that the first filter and the second filter are spaced apart from each other. In various Aspects of the disclosure, the filter assembly includes a splash guard coupled to the first support member and extending from the first support member in a direction opposite from the housing, the splash guard defining an inlet and an outlet. In various Aspects of the disclosure, the splash guard has a conical shape, wherein the outlet of the splash guard is wider than the inlet of the splash guard.

In various Aspects of the disclosure, the canister port is communicatively coupled to a vacuum configured to maintain airflow through the filter system. In various Aspects of the disclosure, the suction canister comprises a soft-sided container. In various Aspects of the disclosure, the suction canister comprises a rigid container. In various Aspects of the disclosure, the filter system is integrated into the port by ultrasonic welding. In various Aspects of the disclosure, the filter system is heat welded to the lid so that the filter system covers the port. In various Aspects of the disclosure, the second filter is hydrophobic. In various Aspects of the disclosure, the first filter comprises a second layer comprised of polymeric filaments less than 6 microns in diameter. In various Aspects of the disclosure, the first filter comprises a second layer comprised of thermoplastic filaments. In various Aspects of the disclosure, the first filter comprises a second layer comprised of polypropylene, polyethylene, polyethylene terephthalate, polyethylene terephthalate co-polymer, or non-thermoplastic filaments. In various Aspects of the disclosure, the first filter comprises an oleophobic first layer. In various Aspects of the disclosure, the first filter defines a specific surface area greater than 0.5 m²/g and less than 2 m²/g. In various Aspects of the disclosure, the second filter comprises a thermoplastic textile layer.

In various Aspects of the disclosure, the port of the housing is configured to be coupled to a vacuum port of a suction canister. In various Aspects of the disclosure, the splash guard has a conical shape, wherein the outlet of the splash guard is wider than the inlet of the splash guard. In various Aspects of the disclosure, the first filter comprises a plurality of layers, and wherein a first layer comprises a different structure than a second layer.

The foregoing embodiments are just that and should not be read to limit or otherwise narrow the scope of any of the inventive concepts otherwise provided by the instant disclosure. While multiple embodiments are disclosed, still other embodiments will become apparent to those skilled in the art from the following detailed description, which shows and describes illustrative embodiments. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 illustrates an exploded view of an embodiment of a filter system including a first filter and a second filter comprising a membrane and a membrane support according to at least one embodiment;

FIG. 2 is a scanning electron micrograph (SEM) of a cross-section of the second filter of FIG. 1, including the membrane and the membrane support according to at least one embodiment;

FIG. 3 is a scanning electron micrograph (SEM) of a cross-section of the first filter of FIG. 1, including a first layer, a second layer, and a third layer of the first filter, each layer defining a thickness according to at least one embodiment;

FIG. 4A is a top view of the filter system of FIG. 1 welded to an example of a lid of a suction canister;

FIG. 4B is a perspective view of the filter system of FIG. 1 welded to a representative lid of a suction canister according to at least one embodiment;

FIG. 5A is a top perspective view of a filter system including a housing and a splash guard according to at least one embodiment;

FIG. 5B is a bottom perspective view of the filter system of FIG. 5A according to at least one embodiment;

FIG. 5C is a cross-sectional view of the filter system of FIG. 5A according to at least one embodiment;

FIG. 5D is an exploded cross-sectional view of the filter system of FIG. 5A according to at least one embodiment;

FIG. 5E is a top-down view of a first support member of the filter system of FIG. 5A according to at least one embodiment;

FIG. 5F is a bottom-up cross-sectional view of the filter system of FIG. 5A, illustrating a second support member of the filter system of FIG. 5A according to at least one embodiment;

FIG. 5G is a top view of a second embodiment of a second support member of the filter system of FIG. 5A according to at least one embodiment;

FIG. 5H is a top view of a third embodiment of a second support member of the filter system of FIG. 5A according to at least one embodiment;

FIG. 6A is a cross-sectional view of a modified embodiment of the filter system of FIG. 5A according to at least one embodiment;

FIG. 6B is a cross-sectional view of a second modified embodiment of the filter system of FIG. 5A according to at least one embodiment;

FIG. 7 is a schematic illustration of the coupling of the filter system of FIG. 5A with a lid of a suction canister according to at least one embodiment;

FIG. 8 is a schematic representation of a test setup for testing the smoke particle retention of a filter system according to at least one embodiment;

FIG. 9 is a graphical illustration of the smoke retention capability of a filter system including the first filter as described herein in comparison with a filter system that does not include the first filter as measured by a percentage of standardized airflow over time with constant smoke exposure according to at least one embodiment;

FIG. 10A is a schematic illustration of the filter system of FIG. 1 coupled to a lid of a representative suction canister according to at least one embodiment;

FIG. 10B is a photograph of the suction canister and filter system of FIG. 10A, where the suction canister is filled with deionized water according to at least one embodiment; and

FIG. 10C illustrates an inverted position of the suction canister of FIG. 10A according to at least one embodiment.

### DETAILED DESCRIPTION

Persons skilled in the art will readily appreciate that various aspects of the present disclosure can be realized by any number of methods and apparatus configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

### Definitions and Terminology

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example. In the event it is determined that individuals having ordinary skill in the relevant arts would not readily ascertain values for such reasonably small differences, the terms “about” and “approximately” can be understood to mean plus or minus 10% of the stated value.

This disclosure is not meant to be read in a restrictive manner. In other words, the terminology used in the application should be read broadly in the context of the meaning those in the field would attribute such terminology.

### Descriptions of the Disclosed Embodiments

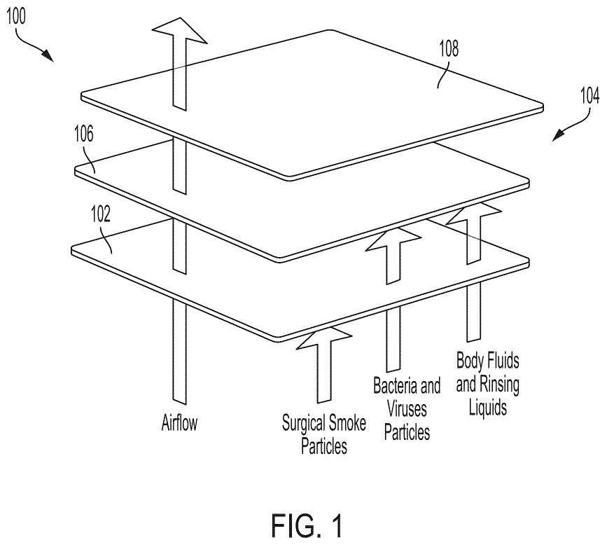

FIG. 1 illustrates a layered filter system 100 for filtering contaminants, such as bodily waste in medical treatment environments, including surgeries. The layered filter system 100 a first filter 102 and a second filter 104, where the second filter 104 may include a membrane 106 and a membrane support 108. The membrane 106 of the second filter 104 is configured to provide a barrier to body fluids, rinsing fluids, and bacteria and viruses, aerosolized or otherwise contained by the fluids, as described further herein. The membrane support 108 offers strength and support to the second filter 104 and may be a weldable material, e.g. polyester non-woven material or thermoplastic, to offer easy integration of the layered filter system 100 with a canister. In some embodiments, the layered filter system 100 may be integrated with, or coupled to, a suction canister lid.

The membrane 106 may be formed of expanded polytetrafluoroethylene (ePTFE), polyethylene, polyvinylidene difluoride, polyethersulfone, or an electrospun polymeric material. The material forming the membrane 106 allows high airflow and has hydrophobic and/or oleophobic characteristics that result in liquid barrier properties. The membrane 106 may be impermeable to, or relatively impermeable to, passage of liquid at maximum operating pressures up to and including 16 psi water entry pressure (WEP), while remaining highly permeable to passage of gases and/or vapor. The membrane 106 may define a thickness of about 40 microns to about 150 microns, where the thickness may be about 40 microns to about 55 microns, about 55 microns to about 70 microns, about 70 microns to about 90 microns, about 90 microns to about 120 microns, or about 120 microns to about 150 microns.

The membrane support 108 includes a textile layer, formed of, for example, a thermoplastic textile, (e.g., a nonwoven polyester), or may otherwise be formed of polyethylene, polypropylene, polyvinyl chloride, or polyethylene terephthalate. The membrane support 108 may define a thickness of about 60 microns to about 600 microns, where the thickness may be about 60 microns to about 110 microns, about 110 microns to about 150 microns, 150 microns to about 190 microns, about 190 microns to about 230 microns, about 230 microns to about 280 microns, about 280 microns to about 330 microns, about 330 microns to about 400 microns, about 400 microns to about 500 microns, or about 500 microns to about 600 microns.

The second filter 104 may define a total thickness from about 100 microns to about 750 microns, where the second filter may have a thickness of about 100 microns to about 200 microns, about 200 microns to about 300 microns, about 400 microns to about 500 microns, about 500 microns to about 600 microns, or about 600 microns to about 750 microns. In various embodiments, the second filter 104 is configured to maintain a liquid entry pressure greater than or equal to 75 kPa for at least 10 minutes at a surface tension of 55.5 mN/m and maintain an airflow of greater than 20 litres/minute at a pressure drop of 11.5 kPa at an active area of 9.3 cm².

In one embodiment, the second filter retains a high liquid retention (i.e., a bubble point at least 8 psi), demonstrates an increase in bacterial filtration efficiency (BFE) and viral filtration efficiency (VFE), and maintains an airflow of about 12 litres/min to about 8 litres/min over an hour of continuous smoke during, for example, electro-surgical procedures. In some embodiments the bubble point may be at least 7.5 psi, at least 8 psi, at least 8.5 psi or from 7 psi to 8.5 psi, from 8 to 9.5 psi, or from 8.5 to 10 psi.

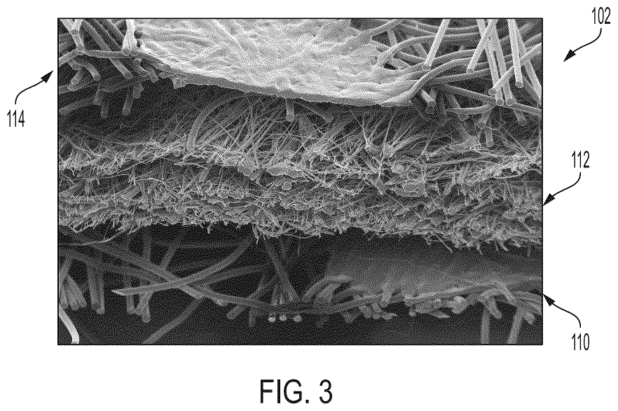

FIG. 3 is microscopic view of a cross-section of the first filter 102. In various embodiments, the first filter 102 defines a specific surface area of about 0.5 m²/g to about 2 m²/g and includes three layers. A first layer 110 of the first filter 102 defines a thickness of about 40 microns to about 200 microns, where the first layer may have a thickness of about 40 microns to about 55 microns, about 55 microns to about 70 microns, about 70 microns to about 100 microns, about 100 microns to about 150 microns, or about 150 microns to about 200 microns. The first filter may be formed of filaments of polypropylene, polyethylene, polyethylene terephthalate, polyethylene terephthalate polyamide co-polymer, polybutylene terephthalate, or combinations thereof. Each filament has a diameter of about 10 microns to about 30 microns. The first layer 110 may have oleophobic characteristics.

A second layer 112 defines a thickness of about 140 microns to about 1600 microns, where the second layer may have a thickness of about 140 microns to about 640 microns, about 640 microns to about 1140 microns, 1140 microns to about 1390 microns, or about 1390 microns to about 1600 microns. The second layer includes a plurality of filaments formed of polypropylene, polyethylene, polyethylene terephthalate, polyethylene terephthalate co-polymer, polybutylene terephthalate, glass, thermoplastic polymers, or non-thermoplastic polymers. Each filament has a diameter of about 0.8 microns to about 8 microns, where each filament may have diameter of about 0.8 microns to about 2 microns, about 2 microns to about 4 microns, or about 4 microns to about 6 microns.

A third layer 114, similar to the first layer 110, defines a thickness of about 40 microns to about 200 microns, where the first layer may have a thickness of about 40 microns to about 55 microns, about 55 microns to about 70 microns, about 70 microns to about 100 microns, about 100 microns to about 150 microns, or about 150 microns to about 200 microns. The first filter may be formed of filaments of polypropylene, polyethylene, polyethylene terephthalate, polyethylene terephthalate polyamide co-polymer, polybutylene terephthalate, or combinations thereof. Each filament has a diameter of about 10 microns to about 30 microns.

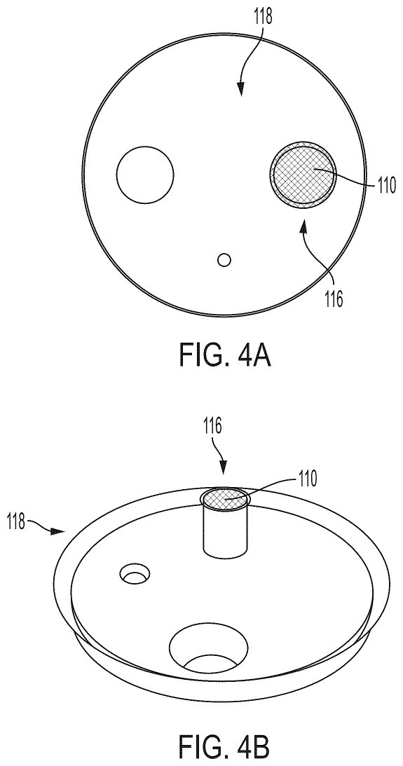

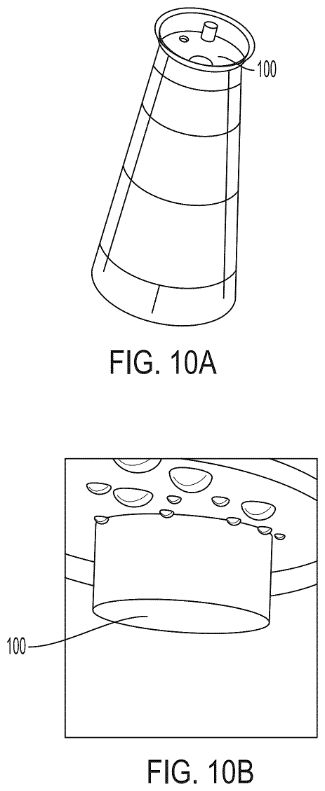

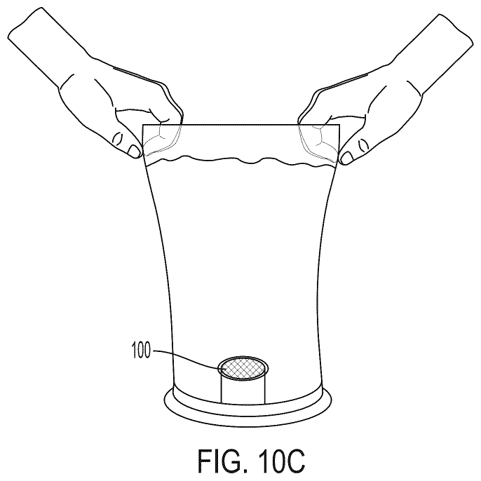

The layered filter system 100 including first filter 102 and second filter 104 described above may be integrated into a vacuum port 116 of a conventional suction canister lid 118 via welding as shown in FIGS. 4A and 4B. This integration method may include a two-step integration, where the second filter 104 (FIG. 1) is heat welded to the canister lid 118 so as to cover the vacuum port 116 of the canister lid 118 in one step, and the first filter 102 is heat welded to the canister lid 118 and the second filter 104 in another step. In other embodiments, the first filter 102 and the second filter 104 may each be welded to the canister lid 118 in a single step so that the second filter 104 is positioned closest to the canister lid 118. As such, in each embodiment, when suction is applied to the vacuum port 116, any media to be filtered first passes through the first filter 102, then through the second filter 104 to exit the vacuum port 116. In some embodiments, the layered filter system 100 may be integrated with the vacuum port 116 via ultrasonic welding or heat welding. The suction canister lid 118 may correspond with representative a suction canisters including a soft-sided container or liner (FIGS. 10A-10C), a cup, rigid container or other relatively resilient-walled container, or both a cup and a liner.

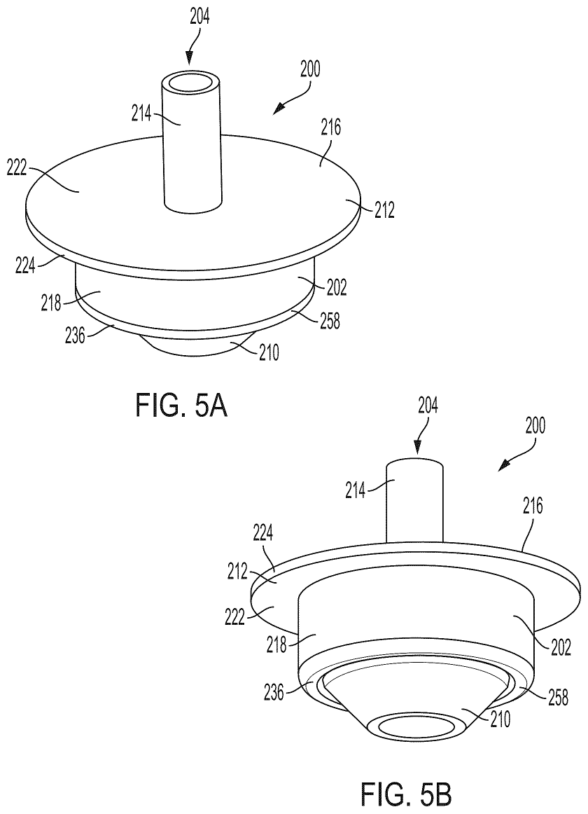

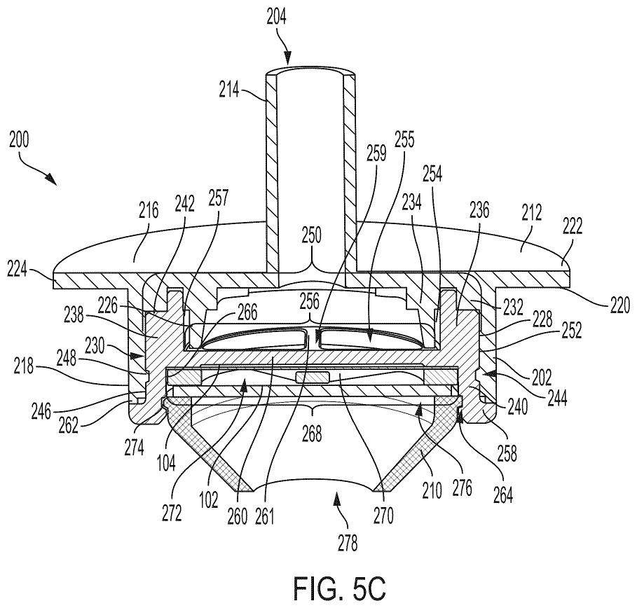

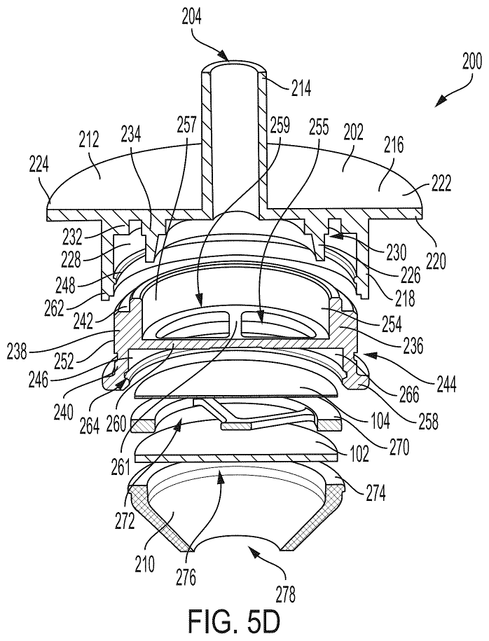

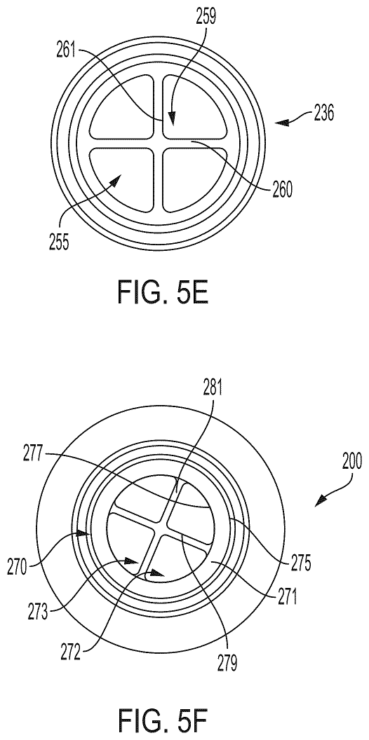

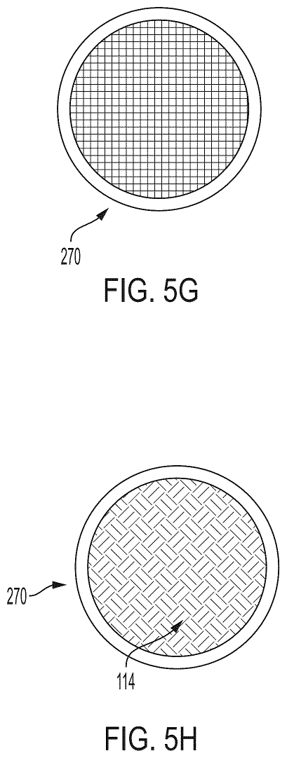

FIGS. 5A-5F illustrate another embodiment of a filter system 200. The filter system 200 includes a housing 202 defining a port outlet 204, a first support member 236, a second support member 270, and a splash guard 210. The housing 202 includes a top portion 212 defining the port outlet 204. The port outlet 204 may further be defined by a port sidewall 214 that extends from a top surface 216 of the top portion 212 in a direction opposite of the splash guard 210 as discussed further herein. A housing sidewall 218 extends from a bottom surface 220 of the top portion 212 in a direction opposite the port sidewall 214. The housing sidewall 218 may be positioned relative to the top portion 212 so that the top portion 212 defines a flange 222 extending outwardly from the housing sidewall 218. The housing sidewall 218 may otherwise be positioned to be flush with an outer perimeter 224 of the top portion 212 or create a larger or smaller flange 222 relative to the housing sidewall 218.

FIGS. 5C and 5D show various components of the filter system 200 in longitudinal section. As shown in FIGS. 5C and 5D, the housing 202 may include an inner wall 226 positioned generally concentrically with the housing sidewall 218. The inner wall 226 and the inner surface 228 of the housing sidewall 218 cooperate to define a coupling gap 230 for assembly of the filter system 200 as discussed further herein. A sidewall step 232 may be positioned on an upper portion of the inner surface 228 of the housing sidewall 218, and one or more inner wall steps 234 may be positioned on one or both sides of an upper portion of the inner wall 226 of the housing 202.

A first support grid, or first support member 236, couples to the housing 202 via the coupling gap 230 as described further herein. The first support member 236 includes an upper sidewall 238 and a lower sidewall 240, each formed to facilitate the assembly of the filter system 200. The upper sidewall 238 is formed into a shape substantially consistent with the coupling gap 230 formed by the housing 202. In some embodiments, the thickness of the upper sidewall 238 is consistent with the general thickness of the coupling gap 230. The upper sidewall 238 may also include a ledge 242 to facilitate a snug fit, or interference fit, with at least one of the sidewall step 232 and the inner wall step 234. As shown in FIGS. 5C and 5D, the upper sidewall 238 may not include ledges corresponding with both of the sidewall step 232 and the inner wall step 234. In other embodiments, the upper sidewall 238 may not include a ledge 242. In still other embodiments, the first support member 236 may couple with the housing 202 via welding or threading. In embodiments where the first support member 236 is coupled to the housing 202 via threading or interference fit, a flexible seal, such as a rubber seal or O-ring, may be positioned between the first support member 236 and the housing 202 to facilitate a fluid-tight seal, where liquid and/or gas may not pass between the first support member 236 and the housing 202.

As shown, an outer surface 252 of the upper sidewall 238 defines an outer diameter 250 sized so that the outer surface 252 contacts the inner surface 228 of the housing sidewall 218 when the first support member 236 is coupled to the housing 202. An inner surface 254 of the upper sidewall 238 defines an inner diameter 256, and an inner perimeter 257, where a grid portion 259 of the first support member 236 spans the area defined by the inner perimeter 257. Referring to FIGS. 5C-5E, the grid portion 259 includes a first arm 260 and a second arm 261 which orthogonally intersect near the center of the first support member 236. In other embodiments, the grid portion 259 may include a greater number of arms to form a checkerboard pattern, a spoked pattern, a Y-shape, or another arrangement or pattern spanning the area defined by the inner perimeter 257. In yet other embodiments, the grid portion 259 may include only a single arm spanning the area defined by the inner perimeter 257. The arms 260, 261 of the grid portion 259 define a plurality of openings 255 within the area defined by the inner perimeter 257 of the first support member 236. The plurality of openings 255 may make up at least 60%, at least 70%, at least 80%, or at least 90% of the area defined by the inner perimeter 257 of the first support member 236.

As shown more clearly in FIG. 5D, a housing groove 244 is defined on the outer surface 246 of the lower sidewall 240 of the first support member 236. The groove 244 corresponds with a ridge 248 positioned on a lower portion of the inner surface 228 of the housing sidewall 218. When coupled, the ridge 248 of the housing 202 is received within the groove 244 of the first support member 236. A rim 258 extends from the outer surface 246 of the lower sidewall 240 along the bottom edge of the lower sidewall 240. When coupled, the rim 258 contacts the bottom edge 262 of the housing sidewall 218 to provide further support to the filter system 200.

A second groove 264 is defined on an inner surface 266 of the lower sidewall 240 to facilitate assembly of the remainder of the filter system 200 as described further herein. The inner surface 266 of the lower sidewall 240 defines a lower diameter 268 for receiving additional components of the filter system 200 as described further herein. The lower diameter 268 may be larger than the inner diameter 256 as shown in FIG. 5C. In other embodiments, the lower diameter 268 may be smaller or substantially the same as the inner diameter 256, as long as the remaining components described further herein are properly received by the lower sidewall 240.

The second filter 104 is positioned beneath the grid portion 259 within lower sidewall 240 of the first support member 236. As shown, the second filter 104 is shaped and sized to fit closely to the inner surface 266 of the lower sidewall 240 and may touch the inner surface 266 of the lower sidewall 240. As such, the second filter 104 may be larger than or at least substantially the same size as the inner diameter 256 of the upper sidewall 238. Preferably, the second filter 104 covers any openings of the first support member 236 defined by the grid portion 259 of the first support member 236 so that airflow through the second filter 104 is largely uninhibited by the grid portion 259 of the first support member 236. The second filter 104 as inserted into the filter system 200 may not include the membrane support 108 (FIG. 1).

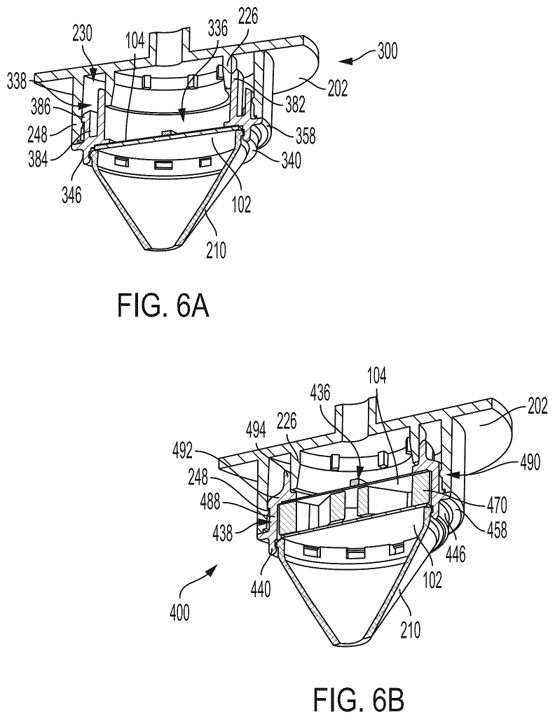

Referring additionally to FIG. 5F, a second support grid, or support member 270 is inserted beneath the second filter 104. The second support member 270 includes a border 271 defining an outer perimeter 275 of the second support member 270 and an inner perimeter 277, the second support member 270 further including a second grid portion 273 spanning the area defined by the inner perimeter 277 of the second support member 270. The second grid portion 273 includes a first arm 279 and a second arm 281 which orthogonally intersect near the center of the second support member 270. In other embodiments, the second grid portion 273 may include a greater number of arms to form a checkerboard pattern as shown in FIG. 5G, which may also apply to the first support member 236 described above, a spoked pattern, or another arrangement or pattern spanning the area defined by the inner perimeter 277 of the second support member 270. In yet other embodiments, the second grid portion 273 may include only a single arm spanning the area defined by the inner perimeter 277. The arms 279, 281 of the second grid portion 273 define a plurality of openings 272 within the area defined by the inner perimeter 277 second support member 270. The plurality of openings may make up at least 60%, at least 70%, at least 80%, or at least 90% of the area defined by the inner perimeter 277. In yet other embodiments, such as the embodiment shown in FIG. 6A and as described further herein, the third layer 114 of the first filter 102 may provide the functionality otherwise provided by the second support member 270 as described herein and illustrated by FIG. 5H.

Referring again to FIGS. 5C and 5D, the second support member 270 is shaped and sized to fit closely to the inner surface 266 of the lower sidewall 240, and may be shaped and sized similarly to the second filter 104 so that the second filter 104 substantially covers the second support member 270 and at least covers any openings 272 defined by the second support member 270. The first filter 102 is positioned beneath the second support member 270. As shown, the first filter 102 may be slightly smaller or substantially the same size as the second support member 270 so that the first filter 102 covers the openings 272 defined by the second support member 270.

The first filter 102 and the second filter 104 are separated by the second support member 270. In other words, the thickness of the second support member 270 provides a distance between the second filter 104 and the first filter 102 so that the second filter 104 and the first filter 102 do not contact each other. The openings 272 defined by the second support member 270 form a liquid reservoir for capturing any liquid that may pass through the first filter 102 and holding said liquid to mitigate saturation of the second filter 104 and leakage of the liquid into the vacuum system. As described above, the second filter 104 is formed from a hydrophobic and/or oleophobic material, coating, or surface treatment to discourage passage of liquid into or through the second filter 104.

The splash guard 210 includes an upper rim 274 configured to be received within the second groove 264 to couple the splash guard 210 to the first support grid 236. The splash guard 210 supports the first filter 102 so that the first filter 102 covers a base opening 276 defined by the upper rim 274 of the splash guard and the first filter 102 is effectively sandwiched between the splash guard 210 and the second support grid 270. As described above, the first filter 102 in turn supports the second support member 270 and the second filter 104. In other words, the splash guard 210 cooperates with the first support member 236 to sandwich the first filter 102, the second support member 270, and the second filter 104 therebetween, holding the first filter 102, the second support member 270, and the second filter 104 in place within the filter system 200.

As shown, the splash guard 210 illustratively may have a frustoconical shape or other shape configured to protect the interior of the filter system 200 from any liquids within the suction canister. In other words, the splash guard 210 can be shaped to mitigate the entry of any unwanted material into the filter system 200 while continuing to facilitate high airflow through the filter system 200. A filter system opening 278 is positioned at the apex of the splash guard 210 to allow entry of air and material which is pulled into the filter system 200 via the vacuum as described above. As shown in FIGS. 5A and 5B, the only components accessible outside of the filter system 200 includes the housing 202, the rim 258 of the first support member 236, and the splash guard 210. The filter system 200 is generally circular corresponding with the vacuum port with which the filter system is integrated as described further herein. In other embodiments, the filter system 200 may be other shapes as needed to integrate with a vacuum port, including oblong, square, rectangular, or other shape.

Now referring to FIGS. 6A and 6B, additional optional embodiments of a filter system are illustrated. In some embodiments, as shown in FIG. 6A, a filter system 300 may not include a second support member 270. In such an embodiment, the second filter 104 and the first filter 102 may be directly stacked together and sandwiched between the splash guard 210 and a first support member 336. A third layer of the first filter 102 as described above may serve a similar spacing or gap function to the second support member 270 in forming a liquid reservoir when a filter system does not include the second support member 270. Regardless of whether formed by the third layer, the second support member 270, or otherwise, in various embodiments of the filter system, the liquid reservoir may have a thickness of between and including 0.04 mm and 80 mm, for example, although a variety of dimensions are contemplated.

In some embodiments, the thickness of the second support member 270 may vary (e.g., creating a reservoir having a variable thickness). In some embodiments, such as filter system 400, the second support member 270 is replaced with a second support member 470 that defines a much greater thickness in comparison to the second supported 270 illustrated in FIG. 5D. The second support member 470 creates a relatively thicker reservoir. As shown, in the modification of FIGS. 6A and 6B, the thickness of a lower sidewall 440 of a first support member 436 directly corresponds with the thickness of the second support member 470, so that the first filter 102, the second filter 104, and the second support member 470 may be snugly sandwiched between the first support member 436 and the splash guard 210.

Additionally, comparing FIGS. 5C, 6A, and 6B and mentioned above, the structure of the first support member 236 may vary to accommodate variations in components of the system and variations in size of included components. In some embodiments, while the coupling mechanism described above to couple the first support member 236 with the housing 202 is generally consistent between the described filter systems 200, 300, and 400, the coupling structure of the first support member 236 may be altered to accommodate the variations described. In some embodiments, the coupling structure of the first support member 236 as shown in FIGS. 5C-5D includes a single upper sidewall 238 corresponding with the coupling gap 230 defined by the housing 202 as described above.

As shown in FIG. 6A, a first support member 336 may include an upper sidewall 338 made up of an inner wall 382 configured to contact the inner wall 226 of the housing 202 and an outer wall 384 having a lip 386 configured to engage with the ridge 248 of the housing 202. The inner wall 382 and the outer wall 384 of the upper sidewall 338 are spaced apart in a manner to fill the coupling gap 230 defined by the housing 202. The first support member 336 further includes a lower sidewall 340 as described above in relation to the first support member 236; however, instead of the rim 358 extending from the outer surface 346 of the lower sidewall 340, the rim 358 extends outwardly from the first support member 336 at a position intermediate of the upper sidewall 338 and the lower sidewall 340 so that the lower sidewall 340 extends below the rim 358 exterior of the housing 202. The remaining structure of the lower sidewall 340, especially in consideration of the coupling mechanism with the splash guard 210, is consistent with the first support member 236 described in connection with the filter system 200.

Now referring to the first support member 436 as shown in FIG. 6B, an upper sidewall 438 has an upside-down L-shape, where the longitudinal portion 488 extends into the coupling gap 230 in contact with the inner surface 228 of the housing sidewall 218, the longitudinal portion 488 defining a groove 490 to engage with the ridge 248 of the housing 202 as described above in relation with the first support member 236. The latitudinal portion 492 extends inwardly from the longitudinal portion 488 and fits beneath the inner wall 226 of the housing 202 while facilitating sandwiching of the second support member 470, the first filter 102, and the second filter 104 with the splash guard 210. An extension 494 extends upward from the latitudinal portion 492 to contact the inner wall 226 so that the upper sidewall 438 spans the coupling gap 230 via the combination of the extension 494 and the longitudinal portion 488 of the upper sidewall 438.

As with the first support member 336 of the filter system 300, in the filter system 400 the first support member 436 further includes a lower sidewall 340 similar to that of the first support member 236; however, instead of a rim 458 extending from an outer surface 446 of the lower sidewall 440, the rim 458 extends outwardly from the first support member 436 at a position intermediate of the upper sidewall 438 and the lower sidewall 440 so that the lower sidewall 440 extends below the rim 458 exterior of the housing 202. The remaining structure of the lower sidewall 440, at least in relation to the coupling mechanism with the splash guard 210, is consistent with the first support member 236 in connection with the filter system 200.

Other variations may made to the filter system 200 beyond those described herein, according to the environment in which the filter system is present and the vacuum system and/or suction canister being utilized so that the filter system is configured to fulfill varying needs or goals depending on each situation. In particular, the thickness of the second support member 270 may vary as discussed above. Additionally, the splash guard may or may not be included with the filter system. In addition to varying design components, assembly methodology may also be varied. In some embodiments, the components of the filter system may be manufactured via injection molding, additive manufacturing, or other known manufacturing methods.

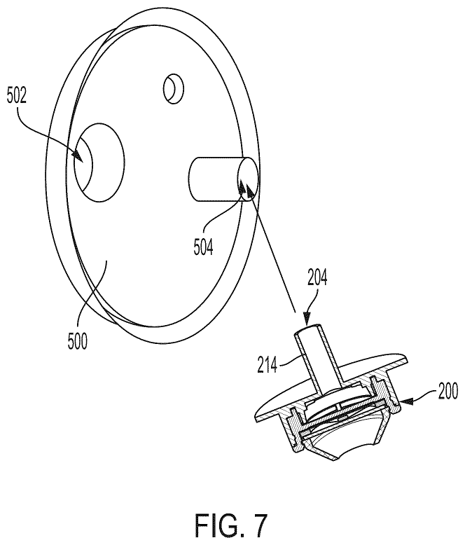

FIG. 7 shows a canister lid 500. The canister lid 500 represents a general canister lid that may encompass several variations of canister lids and suction canisters. The canister lid 500 includes a patient port 502 configured to fluidly couple the lid 500 to a patient via medical tubing (not shown). The canister lid 500 further includes a vacuum port 504 configured to fluidly couple the lid 500 to the vacuum system as described above. As illustrated by FIG. 7, the port sidewall 214 of the filter system 200 is configured to be received by the vacuum port 504 so that the port outlet 204 is placed into fluid communication with the vacuum port 504 and the vacuum system when in operation. The port sidewall 214 illustratively defines a shape and size similar to the vacuum port 504 so that the port sidewall 214 forms an interference fit or otherwise snug fit within the vacuum port 504 so that any airflow created by the vacuum system must pass through the filter system 200 before exiting the lid 500 and passing into the vacuum system.

### Test Methods

### Bubble Point Pressure

Bubble point pressures were measured according to f ASTM F31 6-02 using a Capillary Flow Porometer (Model 3Gzh from Quantachrome Instruments, Boynton Beach, Florida). The sample membrane may be placed into a sample chamber and wetted with Silwick Silicone Fluid (available from Porous Materials Inc.) having a surface tension of 20.1 dynes/cm. The bottom clamp of the sample chamber had a 2.54 cm diameter and a porous metal disc insert defining a thickness of 0.159 cm to support the membrane (Quantachrome part number 75461 stainless steel filter).

### Smoke Retention

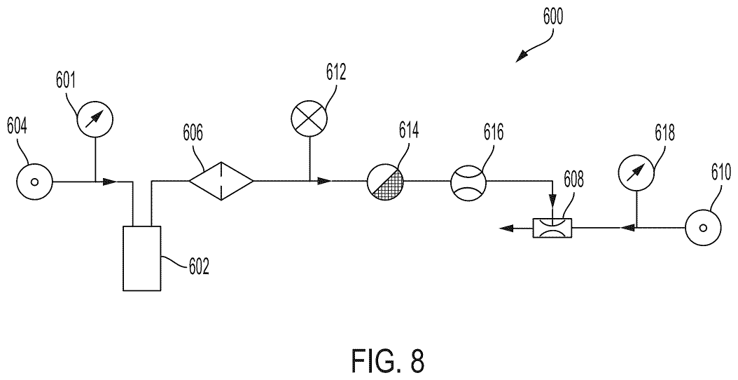

Smoke retention while maintaining high airflow under continuous aerosol burden was tested using the experimental setup 600 illustrated by the schematic representation illustrated in FIG. 8. The experimental setup 600 included an aerosol generator 602 with an inlet that was connected to a compressed air supply 604 via a first pressure regulator 601. An outlet of the aerosol generator 602 was connected to a tested filter system 606 having an active filter area of 7 cm². The aerosol generator 602 was set to 60 psi using pressure regulator 1. The aerosol generator 602 generated mineral oil aerosols in the range of 1.8 μm to 2.0 μm depending on the positive system pressure as shown in Table 1, which includes data generated by Mesa Laboratories Inc., 10 Park Place, Butler, NJ 07405, U.S.A.

TABLE 1
Generalized Output Data for a Colllison Nebulizer from CH Technologies (USA)

| | | Liquid Use Rates (ml/hr) | | | | Air Flow Rates (LPM) | | | |
|---|---|---|---|---|---|---|---|---|---|
| | | | | NSF | | | | NSF | |
| Air | Droplet | | | and 6 | | | | and 6 | |
| Pressure | MMD | 1 jet | 3 jet | jet | 24 jet | 1 jet | 3 jet | jet | 24 jet |
| psig | μm | CN241 | CN24 | CN25 | CN40 | CN241 | CN24 | CN25 | CN40 |
| 20 | 2 | 1.5 | 4.5 | 9.0 | 36.0 | 2.0 | 6.0 | 12.0 | 48.0 |
| 40 | 1.9 | 2.8 | 8.3 | 16.5 | 66.0 | 3.3 | 10.0 | 20.0 | 80.0 |
| 60 | 1.8 | 3.6 | 10.8 | 21.5 | | 4.5 | 13.5 | 27.0 | |
| 80 | 1.75 | 4.3 | 12.8 | 25.5 | | 5.8 | 17.3 | 34.5 | |
| 100 | 1.65 | 4.7 | 14.1 | 28.2 | | 7.0 | 21.0 | 42.0 | |

A Venturi nozzle 608 was connected to the tested filter system 606 via a pressure sensor 612, a water trap 614, and a bidirectional airflow sensor 616. The Venturi nozzle 608 was pressurized with compressed air from a second compressed air supply 610, which generated a vacuum. A second pressure regulator 618 was used to set the vacuum pressure to −50 kPa. The aerosols generated by the aerosol generator 602 were drawn through the tested filter system 606 by the vacuum generated by the Venturi nozzle 608. The tested filter system 606 was subjected to the vacuum pressure and aerosol treatment over 60 minutes. The airflow through the tested filter system 606 was measured with the bidirectional airflow sensor 616, while the pressure sensor 612 was used for monitoring the vacuum pressure and to monitor the pressure exiting the tested filter system 606.

### Viral Filtration Efficiency

A viral filtration efficiency (“VFE”) test was performed using MS-2 Coliphage viruses. MS-2 Coliphage is an unenveloped, single-stranded RNA model virus measuring approximately 23 nm in diameter with a molecular weight of 3.6×10⁶ Daltons. Notably, an MS-2 coliphage virion is relatively smaller in size than a Zika virion, a SARS-COV-2 virion, an HIV virion, a T4 Bacteriophage virion, and a Mimivirus virion. The VFE test was conducted with a challenge load of >1×10⁸ plaque forming units and ≥90% relative humidity to promote cellular viability throughout the test. A MS-2 Coliphage suspension was aerosolized and introduced to a filter system having a first filter and a second filter as disclosed herein. The concentration of MS-2 Coliphage in the aerosolized solution was measured before and after passage through the filter system and compared.

### Bacterial Filtration Efficiency

A bacterial filtration efficiency (“BFE”) test was performed using Brevundimonas diminuta bacteria, a gram-negative bacteria measuring between 0.4 μm and 1.0 μm in diameter. A Brevundimonas diminuta bacterium is relatively smaller in size than a Bacillus bacterium, a PM2.5 bacterium, a red blood cell, and a PM10 bacterium. The BFE test was conducted with a challenge load of >1×10⁸ colony forming units and ≥90% relative humidity to promote cellular viability throughout the test. A Brevundimonas diminuta suspension was aerosolized and introduced to a filter system having a first filter and a second filter as disclosed herein. The concentration of Brevundimonas dimunita in the aerosolized solution was measured before and after passage through the filter system and compared.

Efficiency of the filter system as described above in the VFE and BFE tests was measured according to the following equation:

$$\% Efficiency = \frac{U p s tream Particle Count - Downstream Particle Count}{U p s tream Particle Count} \times 100$$

Additional information on the test methods used in measurement of VFE and BFE as described herein can be found in PDA Journal of Pharmaceutical Science and Technology, January/February 2005, Technical Report No. 40, Sterilizing Filtration of Gases, Section 7.2.4 Challenge Test Methods, which is hereby incorporated by reference.

### Water Entry Pressure

A water entry pressure test was performed using a Mullen RTM Tester (Serial No.: 8240+92+2949, manufactured by BF Perkins, Chicopee, MA, USA) to measure the water intrusion through a membrane layer as defined herein. A test sample of the membrane layer as clamped between a pair of testing fixtures made with square plexiglass sheets defining a thickness of 1.27 cm and a length of 10.16 cm on each side. The lower fixture had the ability to pressurize a section of the sample with water. A piece of pH paper was placed on top of the sample to serve as an indicator of evidence for water entry. The sample was pressurized in small increments of pressure until the pH paper experienced a color change. The corresponding breakthrough pressure or entry pressure was recorded as the water entry pressure, and the average of the three measurements was also recorded.

### EXAMPLES

### Example 1

The membrane 106 of the layered filter systems 100, 200 were subjected to a bubble point test as described above. Conventional filters, labeled in Table 2 below as “Sintered PE1”, “Sintered PE2”, and “Sintered PE 3” were also tested. As shown in Table 2, the bubble point of the conventional filters were below the detection limit of the test equipment, and were therefore recorded as <1 psi. The membrane 106 had a recorded bubble point of 8.5 psi.

TABLE 2
Results of bubble point test for disclosed
membrane and conventional filters

| | Membrane | Conventional Suction Filter Materials | | |
|---|---|---|---|---|
| | 106 Layer | Sintered PE1 | Sintered PE2 | Sintered PE3 |
| Bubble Point (psi) | 8.5 | <1 | <1 | <1 |

The success of the bubble point test indicates that the filter system 100, 200 as described herein is configured to keep surgical waste, including blood and rinsing fluids, contained within a surgical canister to facilitate protection of surgical equipment, hospital personnel, hospital environments, and patients from contamination during procedures in which such surgical canisters are used.

### Example 2

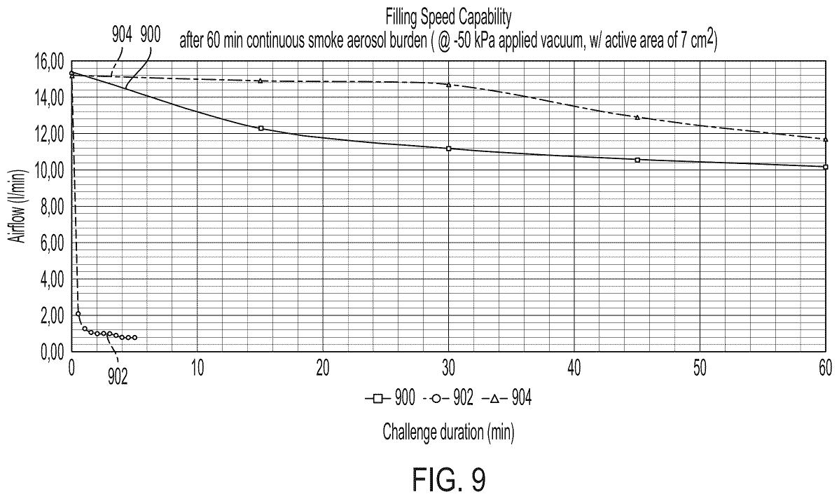

Now referring to FIG. 109, the smoke retention of various filter systems was tested as described above according to the specifications provided in FIG. 9. The filter system was subjected to a vacuum pressure of −50 kPa for an hour, where the vacuum pressure caused interaction of aerosol particles with the filter system.

As shown in FIG. 9, line 902 indicates that a membrane-only embodiment 106 is capable of preventing passage of surgical smoke particles, but airflow dropped significantly within the first 6 minutes of the testing window from about 15.5 litres/min to less than 1 litres/min until continuous surgical suction was no longer possible. Line 900 indicates the performance of a filter system including a membrane 106 and a first filter 102 of the present disclosure. As shown, the drop in airflow under a continuous aerosol burden was significantly lower with the addition of a first filter. The airflow dropped from about 15.5 litres/min to about 12.3 litres/min in the first 15 minutes of the testing window, to about 11.2 litres/min in the first 30 minutes of the testing window, to about 10.5 litres/min in the first 45 minutes of the testing window, and to about 10.2 litre/min at the end of the testing window.

Line 904 indicates the performance of a filter system including a membrane 106 and two layers of a first filter 102 of the present disclosure. The drop in airflow under a continuous aerosol burden was significantly lower with the addition of a second first filter. The airflow dropped from about 15.5 litres/min to about 14.8 litres/min in the first 15 minutes of the testing window, to about 14.7 litres/min in the first 30 minutes of the testing window, to about 12.8 litres/min in the first 45 minutes of the testing window, and to about 13.7 litres/min at the end of the testing window.

Conventional filters (e.g. sintered polyethylene filters) were also tested but were found to not hold back surgical smoke particles. After an hour of continuous aerosol burden as described above, a thin film of oil was detected on the exit of the sintered polyethylene filter and on top of a corresponding bacterial filter, showing that the aerosol was able to pass through the conventional filter. Since aerosols can easily pass through the conventional filter, the filter does not clog or result in a decrease of original airflow. This oil film was not observed at the exit of the filter system including the membrane 106 or the first filter 102 described above. The success of the smoke retention test indicates that the filter system including a first filter as disclosed herein is capable of being used within a surgical smoke environment for at least an hour while maintaining a majority of the maximum possible airflow rate.

### Example 3

The VFE test as described above provided results of a minimum viral filtration efficiency of up to 99.99999%, or Log Reduction Value of 7. This demonstrates an improvement in filtration capabilities over conventional filters, which may only provide results of a minimum viral filtration efficiency of up to 99.99%, or Log Reduction Value of 4, as claimed by the surgical suction container manufacturer, whereas some tests indicate conventional filters provide results of a minimum viral filtration efficiency of up to 98%.

### Example 4

The BFE test as described above provided results of a minimum bacterial filtration efficiency of up to 99.99999%, or Log Reduction Value of 7. This demonstrates improvement in filtration capabilities over conventional filters, which may only provide results of a minimum bacterial filtration efficiency of up to 99.99%, or Log Reduction Value of 4, as claimed by the surgical suction container manufacturer, whereas some tests indicate conventional filters provide results of a minimum viral filtration efficiency of up to 98%.

### Example 5

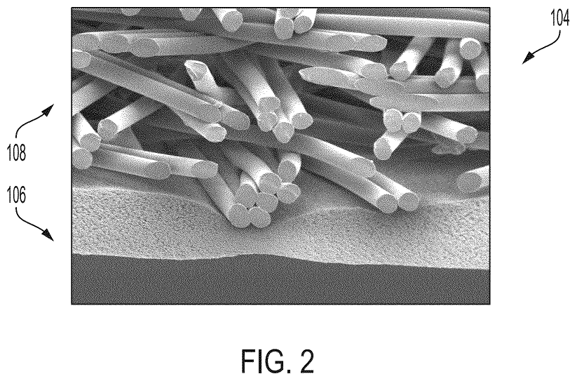

A layered filter system 100 as described above was welded to a suction canister lid to cover the vacuum port of the suction canister lid, and the suction canister lid was coupled with a liner as shown in FIG. 10A to create a lid and liner assembly, where the lid and liner assembly was filled with deionized water as shown in FIG. 10B. Referring again briefly to FIGS. 1-3, the filter system 100 includes a first filter 102 and a second filter 104, where the first filter 102 defines a specific surface area greater than 0.5 m²/g and less than 2 m²/g and includes three layers and where the second filter 104 includes a membrane 106, such as an ePTFE membrane or a membrane comprising electrospun polymers, and a textile membrane support 108 that may be formed from a thermoplastic textile. The second filter 104 defines a total thickness of 230 microns, and is configured to maintain a liquid entry pressure greater than or equal to 75 kPa for at least 10 minutes at a surface tension of 55.5 mN/m and maintain an airflow of greater than 20 litres/min at a pressure drop of 11.5 kPa at an active area of 9.3 cm².

A first layer 110 of the first filter 102 has oleophobic characteristics and defines a thickness of about 80 microns to about 200 microns and has a plurality of filaments, each filament having a diameter of about 17 microns to about 25 microns. A second layer 112 defines a thickness of about 320 microns and has a plurality of filaments, each filament having a diameter of about 3 microns to about 6 microns, where the second layer 112 may have thermoplastic filaments, polypropylene, polyethylene, polyethylene terephthalate, polyethylene terephthalate co-polymer, or non-thermoplastic filaments. The third layer is similar to the first layer in terms of thickness and composition.

Referring again to FIGS. 10A-10C, the lid and liner assembly was placed in a rigid container and coupled to a vacuum pump (Medela Pump “Dominant Flex”), and the vacuum pump was operated at a maximum operation setting for 11 minutes at −88 kPa. No liquid leakage was observed even after continuous liquid contact with the filter system for 11 minutes. In a similar experiment, the lid and liner assembly was inverted overnight in a position illustrated by FIG. 10C. In another experiment, the lid and liner assembly was subjected to another liquid inversion test over eight hours, where the lid and liner assembly was subjected to an extended water entry pressure test at 10 kPa differential pressure. No liquid leakage was observed in either of these experiments.

While the illustrative embodiment includes a filter system for use with a suction canister or suction bag, in other embodiments, the filter systems described above may be utilized in other applications where such filtering is needed, such as in manufacturing of masks or filtering of environmental systems. In other words, the invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A filter system, comprising:
a housing defining an interior chamber with a chamber opening and an outlet port disposed on a base opposite of the chamber opening;
a first support member coupled to the housing;
a first filter positioned over the chamber opening of the housing; and
a second filter positioned between the first support grid and the first filter, wherein a liquid reservoir is defined by the first filter and the second filter.

2. The filter system of claim 1, wherein the second filter includes a liquid entry pressure greater than or equal to 75 kPa for 10 minutes at a surface tension of 55.5 mN/m and maintain an airflow of greater than 20 litres/min at a pressure drop of 11.5 kPa at an active area of 9.3 cm².

3. The filter system of claim 1, wherein the liquid reservoir has a thickness from 0.04 millimeters to 15 millimeters.

4. (canceled)

5. The filter system of claim 1, wherein the liquid reservoir has a thickness from 15 millimeters to 80 millimeters.

6. (canceled)

7. The filter system of claim 1, wherein the liquid reservoir comprises a third layer of the first filter.

8. The filter system of claim 1, wherein the liquid reservoir comprises a second support member positioned between the first filter and the second filter so that the first filter and the second filter are spaced apart from each other.

9. The filter system of claim 1, further comprising a splash guard coupled to the first support member and extending from the first support member in a direction opposite from the housing, the splash guard defining an inlet and an outlet.

10. The filter system of claim 9, wherein the splash guard has a conical shape, wherein the outlet of the splash guard is wider than the inlet of the splash guard.

11. A filtering assembly, comprising:
a lid defining a port; and
a filter system integrated into the port, the filter system comprising:
a first filter defining a thickness of about 120 microns to about 2000 microns, the first filter comprising a first layer and a second layer, wherein the first layer is comprised of a different material than the second layer; and
a second filter having a liquid entry pressure greater than or equal to 75 kPa for 10 minutes at a surface tension of 55.5 mN/m and maintain an airflow of greater than 20 litres/min at a pressure drop of 11.5 kPa at an active area of 9.3 cm².

12. The filtering assembly of claim 11, wherein the canister port is communicatively coupled to a vacuum configured to maintain airflow through the filter system.

13. (canceled)

14. (canceled)

15. The filtering assembly of claim 11, wherein the filter system is fixedly coupled to the lid via welding into or over the port.

16. (canceled)

17. The filtering assembly of claim 11, wherein the second filter is hydrophobic.

18. The filter system of claim 11, wherein the first filter comprises a second layer comprised of polymeric filaments less than 6 microns in diameter.

19. The filter system of claim 11, wherein the first filter comprises a second layer comprised of thermoplastic filaments.

20. The filter system of claim 11, wherein the first filter comprises a second layer comprised of polypropylene, polyethylene, polyethylene terephthalate, polyethylene terephthalate co-polymer, or non-thermoplastic filaments.

21. The filter system of claim 11, wherein the first filter comprises an oleophobic first layer.

22. The filter system of claim 11, wherein the first filter defines a specific surface area greater than 0.5 m²/g and less than 2 m²/g.

23. The filter system of claim 11, wherein the second filter comprises a thermoplastic textile layer.

24. A filter system comprising:
a housing including a base, a port extending from a first side of the base and defining an outlet, a first wall extending from a second side of the base, and a second wall extending from the second side of the base, the first wall and the second wall defining a groove;
a first support member including an upper sidewall configured to be received within the groove of the housing to couple the support member to the housing;
a splash guard coupled to the first support member, the splash guard defining an inlet and an outlet;
a first filter positioned at the outlet of the splash guard;
a second support member positioned between the first filter and the first support member; and
a second filter positioned between the first support member and the second support member.

25. The filter system of claim 25, wherein the port of the housing is configured to be coupled to a vacuum port of a suction canister.

26. (canceled)

27. The filter system of claim 25, wherein the first filter comprises a plurality of layers, and wherein a first layer comprises a different structure than a second layer.
